(19) United States
(12) Patent Application Publication
(10) Pub. No.: US 2024/0006047 A1
(43) Pub. Date: Jan. 4, 2024

(54) REHABILITATION APPROACH PROPOSAL APPARATUS, REHABILITATION APPROACH PROPOSAL METHOD, AND NON-TRANSITORY COMPUTER-READABLE MEDIUM

(71) Applicant: NEC Corporation, Minato-ku, Tokyo (JP)

(72) Inventors: Kosuke Nishihara, Tokyo (JP); Shinichi Daikuya, Osaka (JP)

(73) Assignee: NEC Corporation, Minato-ku, Tokyo (JP)

(21) Appl. No.: 18/039,609

(22) PCT Filed: Oct. 22, 2021
(86) PCT No.: PCT/JP2021/039100

§ 371 Date: May 31, 2023

(30) Foreign Application Priority Data
Dec. 2, 2020 (JP) 2020-200133

(51) Int. Cl. G16H 20/30; A61H 1/02; A63B 24/00

(52) U.S. Cl. CPC G16H 20/30; A61H 1/02; A63B 24/0062

(57) ABSTRACT

A rehabilitation approach proposal apparatus according an example embodiment includes: a bodily state information input unit configured to input bodily state information of a person to be undergoing rehabilitation as bodily state information, the bodily state information being expressed by current states and tilts of external body parts of the person to be undergoing rehabilitation; a desired outcome information input unit configured to select and input a desired outcome of rehabilitation from a plurality of desired outcome candidates as desired outcome information of the person to be undergoing rehabilitation; and an approach determining unit configured to determine a rehabilitation approach to be taken by the person to be undergoing rehabilitation based on the bodily state information and the desired outcome information.

### TECHNICAL FIELD

The present disclosure relates to a rehabilitation approach proposal apparatus, a rehabilitation approach proposal method, and a non-temporary computer-readable medium.

### BACKGROUND ART

Aging population in society is increasing and the number of elderly people in need of nursing care is increasing. In 2025, the baby boomers will reach the age of 75, and a super-aging society will become a reality. The number of users of nursing care services is expected to increase from the age of over 75, and the demand for nursing care is expected to increase in the future.

In order to prevent falling into a state of need for nursing care or to prevent further impairment in the cognitive and physical health, it is a common practice to undergo rehabilitation performed by rehabilitation (rehab) care professionals. Rehabilitation by rehab care professionals refers to physical or operational therapy performed by rehab care professionals for people at a rehabilitation facility or at home. Since due to aging, the elderly people gradually falls into a state in which nursing care is needed, it is necessary to make continuous efforts to look after elderly people.

Proper rehabilitation requires specialized knowledge and experience, including adequately assessing the level of nursing care needed, creating an appropriate rehabilitation plan depending on the bodily state, and providing appropriate rehabilitation. Methods for assessing the level of nursing care needed and the state of bodily function of elderly people include a system for classifying the elderly people by their level of nursing care needed from the support required levels 1, 2 and the care required levels 1 to 5, the Functional Independence Measure (FIM), in which functional independence is rated on a scale of 7 for 18 checkpoints, and performing a test for a range of motion, in which the range of motion of the body joints is measured. Upon checking these states of an individual elderly and at the request of the individual, goals to be achieved are set, tasks to be performed are set, and specific approach for attaining the goals are determined. Moreover, it goes without saying that it is important to perform rehabilitation which is relevant to the actual approach to be taken.

Patent Literature 1 discloses a rehabilitation plan creation system that accepts input of patient's current capability information for identifying the patient's current rehabilitative capability for each factor, patient's current rehabilitative capability information for identifying the patient's current rehabilitative capability for each rehabilitative capability factor, and achievement pattern information for identifying the achievement pattern for attaining the goals set in the rehabilitation plan. Such a rehabilitation planning system identifies, for each rehabilitative capability factor, the difference between the rehabilitative capability required to accomplish the target achievement pattern and the patient's current rehabilitative capability to accomplish the target achievement pattern in a corresponding relationship with the patient's current rehabilitative care ability information and the achievement pattern information, selects the difference to be resolved and rehabilitation plan candidates for resolving the difference, and determines a rehabilitation candidate plan from the rehabilitation plan candidates that can be accomplished with the patient's rehabilitative care ability.

Patent Literature 2 describes that when a first evaluation index calculated based on the motion information at the trainee's facility and the motion information and a second evaluation index at another facility received from a database are in different formats, the received motion information is used to convert the second evaluation index into the format of the first evaluation index, and a rehabilitation plan is created based on the comparison results of the first and second evaluation indices in the same format.

### CITATION LIST

### Patent Literature

Patent Literature 1: Japanese Unexamined Patent Application Publication No. 2011-243203

Patent Literature 2: Japanese Unexamined Patent Application Publication No. 2015-159935

### SUMMARY OF INVENTION

The time available for elderly people to get rehab is limited, and the elderly who are not staying at specialized facilities spend much of their time at home, even if they are undergoing nursing care at rehab facilities. Therefore, how they spend their time at home is important to prevent impairment in their state of health. However, it is not practical for rehab care professionals to be always present. If family members who spend time together at home, care workers such as helpers, and staff at the facilities the elderly people regularly attend can implement appropriate measures to take care of the elderly, they can contribute to prevention of impairment in the elderly people's physical heath.

However, assessing the level of nursing care needed requires experience, and setting up of a rehabilitation plan is not easy, so it is difficult for family members without knowledge or experience in rehab care and for helpers and facility staff member (not engaged in rehabilitation profession), to implement appropriate measures, and moreover, performing wrong therapy can cause adverse effects. In Patent Document 1, information on patient's current physical function and information on patient's current capability to get rehabilitation are entered, but how to assess these information that are necessary to ascertain the level of nursing care needed is not described. In addition, Patent Document 2 only describes BI (Batheal Index) and FIM (Functional Independence Measure) as measures to assess the activities of daily living (ADL) of trainees.

An object of the present disclosure is to provide, in view of the aforementioned problem, a rehabilitation approach proposal apparatus, a rehabilitation approach proposal method, and a non-temporary computer-readable medium, each adapted to determine a suitable approach to be taken by a person to be undergoing rehabilitation.

According to an aspect of the present disclosure, a rehabilitation approach proposal apparatus includes: bodily state information input means for inputting bodily state information of a person to be undergoing rehabilitation as bodily state information, the bodily state information being expressed by current states and tilts of external body parts of the person to be undergoing rehabilitation; desired outcome information input means for selecting and inputting a desired outcome of rehabilitation from a plurality of desired outcome candidates as desired outcome information of the person to be undergoing rehabilitation; and approach determining means for determining a rehabilitation approach to be taken by the person to be undergoing rehabilitation based on the bodily state information and the desired outcome information.

According to an aspect of the present disclosure, a rehabilitation approach proposal method includes determining a rehabilitation approach to be taken by a person to be undergoing rehabilitation based on bodily state information of the person to be undergoing rehabilitation and desired outcome information, the body state information being expressed by current states and tilts of external body parts of the person to be undergoing rehabilitation and the desired outcome information being selected from a plurality of desired outcome candidates.

According to an aspect of the present disclosure, a rehabilitation approach proposal program causes a computer to execute processes for determining a rehabilitation approach to be taken by a person to be undergoing rehabilitation based on bodily state information and desired outcome information, the body state information being expressed by current states and tilts of external body parts of the person to be undergoing rehabilitation and the desired outcome information being selected from a plurality of desired outcome candidates.

According to the present disclosure, a rehabilitation approach proposal apparatus, a rehabilitation approach proposal method, and a rehabilitation approach proposal program each adapted to determine a suitable approach to be taken by a person to be undergoing rehabilitation are provided.

### BRIEF DESCRIPTION OF DRAWINGS

FIG. 1 is a block diagram showing a configuration of a rehabilitation approach proposal apparatus according to an example embodiment;

FIG. 2 is a block diagram showing a configuration of the rehabilitation approach proposal apparatus according to an Example;

FIG. 3 is a diagram showing an example of desired outcome candidates, goal candidates, and task candidates for a person to be undergoing rehabilitation;

FIG. 4 is a diagram showing an example of approach candidates for the determined task candidates;

FIG. 5 is a flowchart for explaining a rehabilitation approach proposal method according to an Example; and

FIG. 6 is a block diagram showing a hardware configuration of a rehabilitation approach proposal apparatus according to an Example.

### EXAMPLE EMBODIMENT

Hereinbelow, specific example embodiments in which the present disclosure is employed will be described with reference to the drawings. However, the present disclosure is not to be limited to any one of the example embodiments described below. Note that the following description and the attached drawings are simplified where appropriate for the sake of clarifying the explanation. In the figures, the identical reference symbols denote identical structural elements and the redundant explanation thereof is omitted.

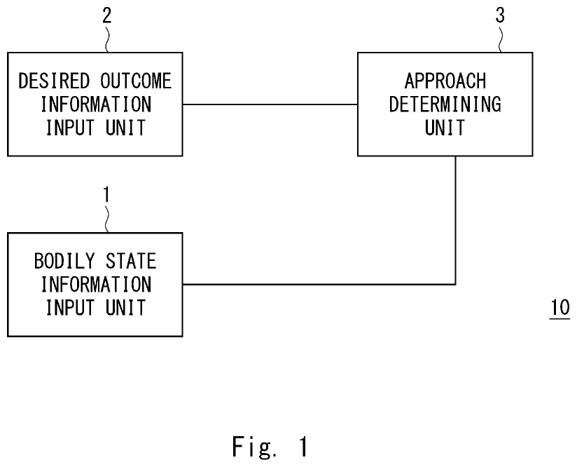

FIG. 1 is a block diagram showing a configuration of a rehabilitation approach proposal apparatus according to an example embodiment. As shown in FIG. 1, a rehabilitation approach proposal apparatus 10 includes a bodily state information input unit 1, a desired outcome information input unit 2, and an approach determining unit 3. The bodily state information input unit 1 expresses the current bodily state of a person to be undergoing rehabilitation in terms of the states and tilt directions of external body parts of the person to be undergoing rehabilitation, and inputs the information as the bodily state information into the approach determining unit 3. The desired outcome information input unit 2 selects desired outcomes from a plurality of desired outcome candidates and inputs the selected desired outcomes into the approach determining unit 3 as desired outcome information of the person to be undergoing rehabilitation. The approach determining unit 3 decides the approach to be taken by the person to be undergoing rehabilitation based on the bodily state information and the desired outcome information.

In this way, the rehabilitation approach proposal apparatus 10 can appropriately assess the level of nursing care a person to be undergoing rehabilitation needs by referring to the bodily state information in which the current bodily state of the person to be undergoing rehabilitation is expressed in terms of the states and the tile directions of the external body parts of the person to be undergoing rehabilitation. Further, the rehabilitation approach proposal apparatus 10 can determine the approach suitable to be taken by the person to be undergoing rehabilitation by referring to the bodily state information and the desired outcome information. Furthermore, based on the determined rehabilitation approach, the rehabilitation approach proposal apparatus 10 can propose, even to any person who does not have any knowledge or experience in providing rehabilitation, a suitable approach that can be taken by the person to be undergoing rehabilitation.

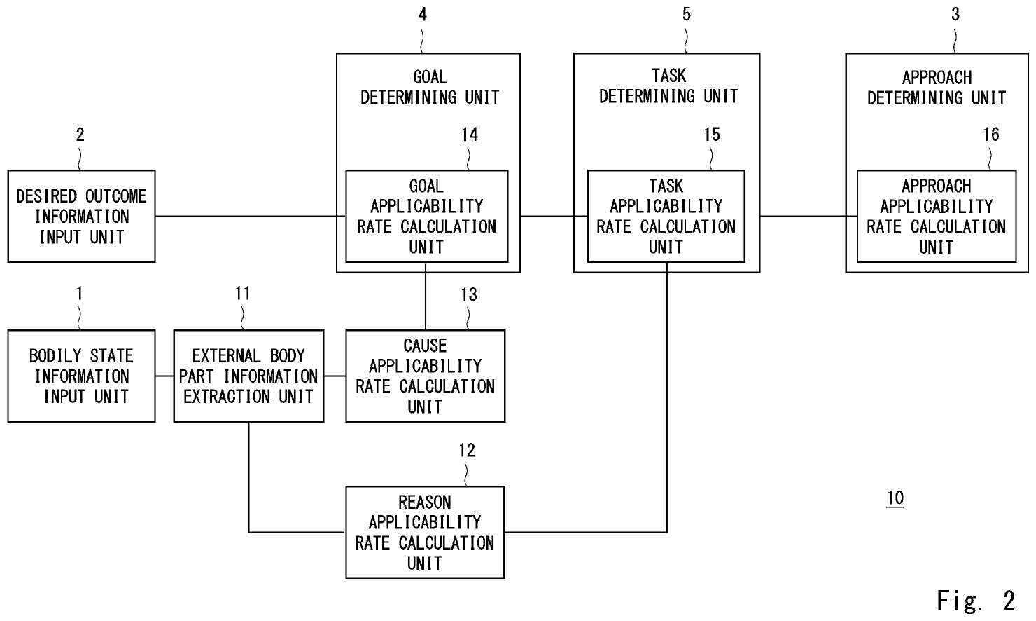

FIG. 2 is a diagram showing a specific configuration of the rehabilitation approach proposal apparatus 10 according to a specific Example. The rehabilitation approach proposal apparatus 10 shown in FIG. 2 includes, in addition to the configuration shown in FIG. 1, a goal determining unit 4, a task determining unit 5, an external body part information extraction unit 11, a cause applicability rate calculation unit 13, and a reason applicability rate calculation unit 12. The goal determining unit 4 and the task determining unit 5 are arranged between the bodily state information input unit 1 and the approach determining unit 3 in this order. The goal determining unit 4 includes a goal applicability rate calculation unit 14. The task determining unit 5 includes a task applicability rate calculation unit 15. Further, the approach determining unit 3 includes an approach applicability rate calculation unit 16.

The bodily state information input from the bodily state information input unit 1 is passed on to the external body part information extraction unit 11, and then the necessary information within the bodily state information is input to the cause applicability rate calculation unit 13 and the goal applicability rate calculation unit 14, respectively. The result of calculation performed by the cause applicability rate calculation unit 13 is used for the calculation performed by the goal determining unit 4, and the result of calculation performed by the reason applicability rate calculation unit 12 is used for the calculation performed by the task determining unit 5. Also, the desired outcome information input from the desired outcome information input unit 2 is passed on to the goal determining unit 4 and the task determining unit 5 for predetermined calculation, and then the result of the calculation is input to the approach determining unit 3. That is, the result of calculation performed based on the bodily state information input from the bodily state information input unit 1 and the desired outcome information input from the desired outcome information input unit 2 is input to the approach determining unit 3. The detailed configuration is described below.

The bodily state information is information indicating the current bodily state of a person to be undergoing rehabilitation in terms of the states and the tilt directions of the external body parts of the person to be undergoing rehabilitation. The bodily state information input unit 1 acquires information about the current bodily state of a person to be undergoing rehabilitation, which is entered by, for example, a caregiver through a user interface. Here, a caregiver refers to a person who supports a person to be undergoing rehabilitation in performing rehabilitation. A caregiver is, for example, a family member of a person to be undergoing rehabilitation, a care worker such as a helper, a staff of a rehab facility, or the like. Further, definition of a caregiver may also include a rehab professional staff member.

The bodily state information input unit 1 may convert the acquired information about the current bodily state of a person to be undergoing rehabilitation into bodily state information. Further, the bodily state information input unit 1 may also acquire bodily state information by having a caregiver directly enter the bodily state information through an input apparatus.

Desired outcome information is information indicating the degree of recovery desired to be achieved by a person to be undergoing rehabilitation after undergoing rehabilitation. Desired outcome information may be information about outcomes to be achieved by a person to be undergoing rehabilitation as desired by a caregiver. The desired outcome information input unit 2 acquires the desired outcome information, which is entered by, for example, a caregiver through a user interface. The desired outcome information input unit 2 can acquire at least one desired outcome candidate selected by a caregiver from a plurality of preset desired outcome candidates as desired outcome information. Alternatively, based on the desired outcome information arbitrarily entered by a caregiver, at least one desired outcome matching the desired outcome information that has been entered may be acquired from a plurality of preset desired outcome candidates as desired outcome information.

The bodily state information input unit 1 expresses the bodily state information by at least one of the 18 external body parts of the person to be undergoing rehabilitation and the tilt of the external body parts in seven directions. The 18 external body parts of the person to be undergoing rehabilitation are, for example, leftside and rightside of the cervical, left and right shoulders, leftside and rightside of the hip, left and right knees, left and right ankles, left and right elbows, left and right wrists, and fingers of the left and right hands. The seven directions indicating the directions in which these external body parts are tilted are the left direction, the right direction, the front direction, the back direction, the upward direction, the downward direction, and null tilting. A plurality of combinations of at least one of the 18 external body parts and the tilt direction of the external body parts defined by the seven directions can be stored in the bodily state information input unit 1 as a plurality of bodily state candidates. A person to be undergoing rehabilitation or a caregiver can select, from the plurality of bodily state candidates, a state that is compatible with the current bodily state of the person to be undergoing rehabilitation.

For the sake of simplifying the description, the external body parts and the tilt thereof are limited in the following description. As an example, description is made taking four external body parts of leftside hip, left knee, rightside hip, and right knee and three tilt directions of the front direction, the back direction, and no tilting. Here, it is assumed that the State 1 shown in Table 1 is selected by the bodily state information input unit 1.

TABLE 1
Example of State

| | |
|---|---|
| State 1 | Front part of leftside hip, back of left knee, |
| | front part of leftside hip, back of right knee |

The external body part information extraction unit 11 extracts, from the bodily state information, cause body part information and reason body part information, the reason body part including at least one of the external body parts included in the cause body parts, and inputs the reason body part information to the reason applicability rate calculation unit 12 and the cause body part information to the cause applicability rate calculation unit 13. Here, the cause body parts refer to a cause for the person to be undergoing rehabilitation to have the current bodily state, and indicate a predetermined combination of external body parts. Causative body parts may be indicated by a combination of external body parts of a person to be undergoing rehabilitation where a problem may be occurring in light of the current bodily state of the person to be undergoing rehabilitation. Further, cause body parts may also be indicated by a combination of a plurality of body parts of interest in setting the goals for a person to be undergoing rehabilitation, which will be described later. Reason body parts refer to external body parts that are causing the person to be undergoing rehabilitation to have the current bodily state. The reason body parts may be indicated by one of the external body parts that may be impaired and causing the current bodily state of the person to be undergoing rehabilitation. Further, the reason body parts may also be indicated by external body parts of interest in setting the tasks to be performed by a person to be undergoing rehabilitation, which will be described later.

The extraction of the cause body parts and reason body parts can be performed, for example, in accordance with predetermined extraction rules. Table 2 shows an example of the rules for extracting the cause body parts and the reason body parts.

TABLE 2
Extraction Rules

| Rule ID | Cause body part | Reason body part |
|---|---|---|
| 1 | leftside hip + left knee | leftside hip, left knee |
| 2 | left knee + right knee | left knee |
| . . . | . . . | . . . |

The extraction rules are defined in advance so as to correspond to the case in which the bodily state information includes information about the specific external body parts and/or the tilt states of the external body parts. At least one extraction rule is predetermined for a set of a combination of a plurality of external body parts and the tilt state of the plurality of external body parts, such as State 1 described above. In Table 2, for example, there are Rule 1 and Rule 2. When there are a plurality of extraction rules as described above, the number of the cause body parts extracted are as many as the number of rules. According to Rule 1, the combination of the external body parts that are cause body parts is “leftside hip+left knee”, and according to Rule 2, the combination of the external body parts that are cause body parts is “left knee+right knee”.

Further, the reason body parts are whole or a part of the external body parts included in the cause body parts. According to Rule 1, the reason body parts are “leftside hip” and “left knee”, which are external body parts of the whole cause body parts. On the other hand, according to Rule 2, the reason body part is “left knee”, which is an external body part of a part of the cause body parts. If State 1 is selected by the bodily state information input unit 1, according to Rule 1, the cause body parts to be extracted are “front part of leftside hip+back of left knee,” and the reason body parts to be extracted are “front part of leftside hip” and “back of left knee”.

The reason applicability rate calculation unit 12 calculates the applicability rate of the possible tilt states of the external body parts for each of the extracted reason body parts. Perceptions as to whether or not the external body parts are tilted differ among those to be undergoing rehabilitation even if their external body parts are tilted in a similar manner. Therefore, in order to compensate for the error in the information about the tilt states of the external body parts that is based on the information input by a caregiver, the applicability rate of the possible tilt states of the external body parts for the extracted reason body parts is calculated. As described above, by referring to the applicability rates of the possible tilt states of the external body parts for the respective extracted reason body parts, it is possible to appropriately assess the level of care the person to be undergoing rehabilitation needs, whereby it is possible to determine effective approach to be taken by the person to be undergoing rehabilitation in undergoing rehabilitation.

In the case of Rule 1, the extracted reason body parts are “front part of left-hip” and “back of left-knee”, and these information are input in the reason applicability rate calculation unit 12. The external body parts of the input reason body parts that are in the possible tilt states are “front part of leftside hip”, “back part of leftside hip”, “no tilting of leftside hip”, “front of left knee”, “back of left knee”, and “no tilting of left knee”.

The applicability rates of the respective tilt states of the reason body parts can be determined according to the tilt states of the extracted reason body parts by referring to the tables given in advance. For example, as shown in Table 3, the applicability rates of the possible tilt states of the external body parts with respect to the extracted tilt states can be set in the reason applicability rate calculation unit 12. Note that in Table 3, the applicability rates of the possible tilt states of the external body parts with respect to the extracted tilt states is set regardless of the external body parts, but it is not limited thereto, and different values may be set for each external body part.

TABLE 3
Applicability rate of possible tilt with respect to extracted tilt

| | | Extracted tilt | | |
|---|---|---|---|---|
| | | Front | Back | No tilt |
| Possible tilt | Front | 80% | 0% | 15% |
| | Back | 0% | 80% | 15% |
| | No tilt | 20% | 20% | 70% |

Referring to Table 3, the applicability rates of the possible tilt states of the reason body parts for the respective extracted reason body parts are determined as shown in Table 4. The calculation of the applicability rates of the tilt states of the reason body parts is performed in accordance with the respective rules. That is, a table like Table 4 will be generated as many as the number of rules.

TABLE 4
Applicability rate of tilt of possible reason body part
with respect to tilt of extracted reason body part

| | | Appli- |
|---|---|---|
| Extracted reason | Possible reason | cability |
| body part | body part | rates |
| Front side of | Front of leftside hip | 80% |
| leftside hip | Back of lefside hip | 0% |
| | No tilting | 20% |
| Back of left knee | Front of left knee | 0% |
| | Back of left knee | 80% |
| | No tilting | 20% |

The cause applicability rate calculation unit 13 calculates the applicability rate of each possible tilt state for the respective cause body parts. As described above, information about the tilt states of the external body parts based on the information about the bodily state of a person to be undergoing rehabilitation entered by a caregiver may be incorrect. To compensate for this potential error, calculation of an applicability rate of a possible tilt state of the respective external body parts is performed for the extracted cause body parts. As described above, by referring to the applicability rates of the possible tilt states of the external body parts for the extracted reason body parts, it is possible to appropriately assess the level of care a person to be undergoing rehabilitation needs, whereby it is possible to determine an effective approach to be taken by a person to be undergoing rehabilitation. In the case of Rule 1, the extracted cause body parts are “front part of leftside hip+back of left knee” and this value is input in the cause applicability rate calculation unit 13. The possible tilt states of the extracted cause body parts according to Rule 1 are as shown in Table 5 under the possible cause body parts.

TABLE 5
Applicability rates of tilt of possible cause body part
with respect to tilt of extracted cause body part

| Extracted cause | | |
|---|---|---|
| body part | Possible cause body part | Applicability rate |
| Front part of | Front part of leftside hip + | 80% × 0% = 0% |
| left hip + back | back of left knee | |
| of left knee | Back part of leftside hip + | 0% × 0% = 0% |
| | front of left knee | |
| | No tilt of leftside hip + | 20% × 0% = 0% |
| | front of left knee | |
| | Front part of leftside hip + | 80% × 80% = 64% |
| | Back part of left knee | |
| | Back part of leftside hip + | 0% × 80% = 0% |
| | back of left left knee | |
| | No tilt of leftside hip + | 20% × 80% = 16% |
| | back of left knee | |
| | Front of leftside hip + | 80% × 20% = 16% |
| | no tilt of left knee | |
| | Back of leftside hip + | 0% × 20% = 0% |
| | no tilt of left knee | |
| | No tilt of lefside hip + | 20% × 20% = 4% |
| | no tilt of left knee | |

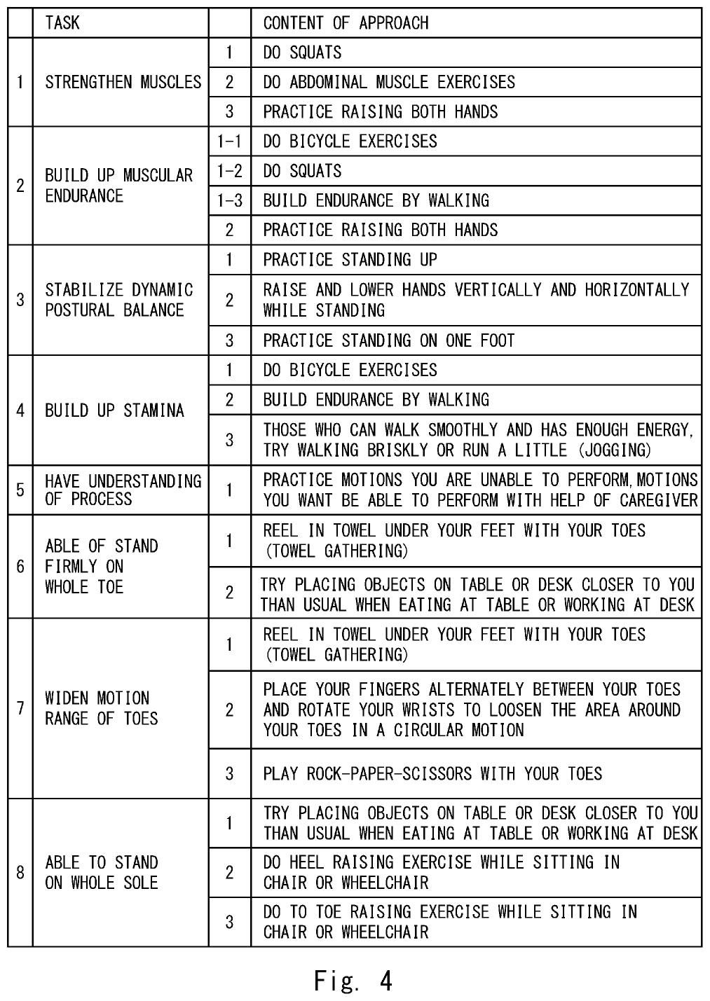

That is, extracted cause body parts refer to one combination of external body parts (e.g. front part of leftside hip+back of left knee) per one rule and the tilt states of each of the external body parts. The possible cause body parts refer to a combination of the external body parts corresponding to the extracted cause body parts and the possible tilt states of the external body parts. Here, the extracted cause body parts are a combination of two external body parts and there are three possible tilt states, and therefore there are 3×3=9 possible cause body parts. The applicability rates of the tilt states of these possible cause body parts can be calculated by multiplying the applicability rates of the tilt states of the respective external body parts shown in FIG. 4. A table like Table 5, showing the cause applicability rates, is also generated as many as the number of rules.

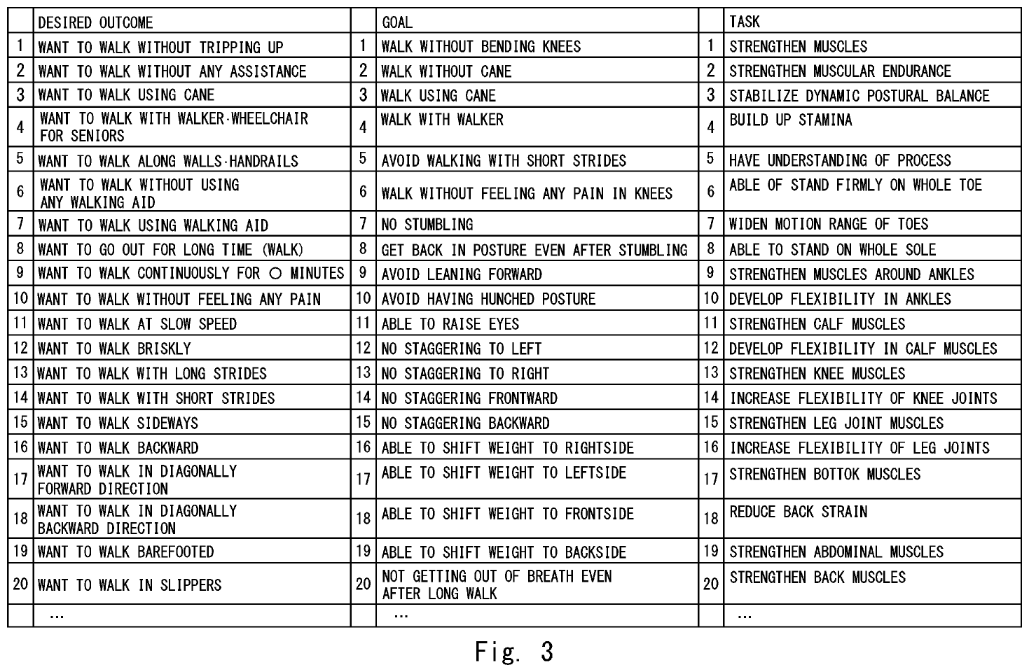

Here, as shown in FIG. 3, the desired outcome candidates, the goal candidates, and the task candidates for a person to be undergoing rehabilitation are set in advance. A person to be undergoing rehabilitation selects, through a user interface, the desired outcome candidates from the desired outcome candidates shown in FIG. 3.

The desired outcome information input unit 2 transmits, to the goal determining unit 4, desired outcome candidates that have been selected from a plurality of desired outcome candidates as desired outcome information of the person to be undergoing rehabilitation. The goal applicability rate calculation unit 14 included in the goal determining unit 4 calculates the applicability rates of the plurality of goal candidates to be achieved by the person to be undergoing rehabilitation using the desired outcome information that has been entered and the applicability rates of the tilt states of the cause body parts calculated by the cause applicability rate calculation unit 13. Examples of the plurality of goal candidates are shown in FIG. 3.

By referring to the goal applicability rate calculation table like Table 6 that is configured in advance, the applicability rates of the candidate goals are calculated as the product of the applicability rates (p₁, p₂, . . . ) allocated to the possible cause body parts corresponding to the respective desired outcomes, the desired outcomes (Outcome 1, Outcome 2 . . . ) that have been input, and the applicability rates of the possible cause body parts. Note that the applicability rates can be 0 for combinations that are not shown in the table. Further, the applicability rates of the desired outcome are 100% for those that are input and 0% otherwise.

TABLE 6
Goal applicability rate calculation table

| | | | | | Appli- |
|---|---|---|---|---|---|
| Rule | Extracted reason | Desired | Possible cause | | cability |
| ID | body part | Outcome | body part | Goal | rate |
| 1 | Leftside hip + | Desired | Front part of | Goal 1 | p₁ |
| | left knee | outcome 1 | leftside hip + | | |
| | | | back of left | | |
| | | | knee | | |
| | | Desired | Front part of | Goal 2 | p₂ |
| | | outcome 2 | leftside hip + | | |
| | | | front of left | | |
| | | | knee | | |
| 2 | Left knee + | Desired | Back of left | Goal 3 | p₃ |
| | right knee | outcome 3 | knee + back | | |
| | | | of right knee | | |
| . . . | . . . | . . . | . . . | . . . | |

The applicability rates (P₁, P₂, . . . ) of the goal candidates according to Rule 1 (see, “goal candidates” in FIG. 3) are as shown in Table 7. In Table 7, if, for example, the desired outcome that has been input is “Outcome 1 (FIG. 3, “to be able to walk without tripping up”)”, the corresponding goal will be Goal 1 and the applicability rate P₁ is calculated. Note that if a plurality of possible cause body parts are involved in achieving one desired outcome, the applicability rate is calculated as the sum of applicability rates of these possible cause body parts. The applicability rates of the goal candidates are calculated also as many as the number of rules.

TABLE 7
Goal applicability rate

| | Corresponding | Corresponding applicable | Applicability |
|---|---|---|---|
| Goal | desired outcome | cause body part | rate |
| Goal 1 | Desired | Front part of lefside hip + | P₁ = 100% × |
| | outcome1 | Back of left knee | 64% × p₁ |
| Goal 2 | Desired | Front part of leftside hip + | P₂ = 0% × |
| | outcome 2 | Front of left knee | 0% × p₂ |
| Goal 3 | None | None | 0% |

The goal determining unit 4 determines one or a plurality of goals among the plurality of goal candidates based on the applicability rates of achieving the goal candidates calculated by the goal applicability rate calculation unit 14. Here, the “goals” are items regarding the states of the external body parts to be achieved by the person to be undergoing rehabilitation so that the person to be undergoing rehabilitation will be in a desired state of the external body parts in the future. The goals may be items regarding the states of the external body parts that are expected to be achieved by improving the states of a plurality of external body parts. The goals can be determined automatically to be, for example, the one goal candidate having the highest applicability rate or N-number (where N is an integer greater than 1) of the goal candidates in the descending order of the applicability rates. Further, across all the rules, the N-number of the goal candidates having the top N-number of applicability rates calculated by the goal applicability rate calculation unit 14 may be displayed to a person to be undergoing rehabilitation through a user interface. A person to be undergoing rehabilitation can select and determine one goal from the proposed goal candidates. Note the value of N can be given in advance, or a threshold value may be defined in advance, and the value of N can be set be to be equal to or greater than the threshold value that is defined in advance.

Note that determination of the goals may not be performed in the goal determining unit 4 and the applicability rates for all the goal candidates may be input in the task determining unit 5 in a latter stage. That is, the applicability rates of the determined goals or the applicability rates of a plurality of goal candidates are input in the task applicability rate calculation unit 15 of the task determining unit 5.

The task determining unit 5 determines the tasks to be performed by the person to be undergoing rehabilitation based on the applicability rates of the determined goals to be achieved or the applicability rates of the goal candidates, and the reason body parts. Here, “tasks” refer to challenges to be overcome by a person to be undergoing rehabilitation in order to attain the goals. Tasks may be challenges expected to be overcome owing to improvement in the function of one external body part. The task applicability rate calculation unit 15 included in the task determining unit 5 calculates the applicability rates of a plurality of task candidates to be performed by a person to be undergoing rehabilitation using the applicability rates of the determined goals to be achieved or the applicability rates of a plurality of goal candidates and the applicability rates of the tilt states of the reason body parts calculated by the reason applicability rate calculation unit 12. Examples of the plurality of task candidates are shown in FIG. 3.

Here, it is assumed that the determined goals have been input in the task applicability rate calculation unit 15. By referring to a task applicability rate calculation table like Table 8 that is configured in advance, the applicability rates of the task candidates are calculated as the product of the applicability rates (q₁, q₂, . . . ) allocated to the possible reason body parts corresponding to the goals, the goals that have been input (Goal 1, Goal 2, . . . ), and the applicability rates of the possible reason body parts. Note that the applicability rates can be zero for combinations that are not shown in the table.

TABLE 8
Task applicability rate calculation table

| | | | | | Appli- |
|---|---|---|---|---|---|
| Rule | | | Possible reason | | cability |
| ID | Cause body part | Goal | body part | Task | rate |
| 1 | Leftside hip + | Goal 1 | Front part of | Task 1 | q₁ |
| | left knee | | leftside hip | | |
| | | Goal 1 | Back of knee | Task 2 | q₂ |
| | | Goal 2 | Front of left knee | Task 3 | q₃ |
| 2 | Left knee + | Goal 3 | Front of left knee | Task 4 | q₄ |
| | right knee | | | | |
| . . . | . . . | . . . | . . . | . . . | . . . |

The applicability rates of the task candidates (Q₁, Q₂, . . . ) according to Rule 1 (see, “task candidates” in FIG. 3) are, for example, those shown in Table 9. In Table 9, if, for example, goal that is input is “Goal 1”, the tasks that correspond to Goal 1 are Task 1 and Task 2, and the respective applicability rates Q₁ and Q₂ are calculated. Note that if a plurality of reason body parts are involved in achieving one goal, the applicability rates can be the sum of the applicability rates of each reason body part. The applicability rates of the task candidates are generated as many as the number of rules.

TABLE 9
Task applicability rate

| | Corresponding | Corresponding possible | |
|---|---|---|---|
| Task | goal | reason body part | Applicability rate |
| Task 1 | Goal 1 | Front part of leftside hip | Q₁ = P₁ × 80% × q₁ |
| Task 2 | Goal 1 | Back part of left knee | Q₂ = P₁ × 80% × q₂ |
| Task 3 | Goal 2 | Front part of left knee | Q₃ = P₂ × 0% × q₃ |
| Task 4 | None | None | 0% |

The task determining unit 5 determines one or a plurality of tasks from the plurality task candidates based on the applicability rates of the task candidates calculated by the task applicability rate calculation unit 15. Note that the applicability rates of the task candidates can be calculated by directly inputting the applicability rates of the plurality of goal candidates calculated by the goal applicability rate calculation unit 14 into the task applicability rate calculation unit 15. In this case, since the applicability rates of a plurality of goal candidates are input, the applicability rates of the task candidates can be the sum or the mean value of the applicability rates of the respective goal candidates calculated.

The tasks can be determined automatically to be, for example, the one task candidate having the highest applicability rate or N-number (where N is an integer greater than 1) of the task candidates in the descending order of the applicability rates. Further, across all the rules, the N-number of the task candidates having the top N-number of applicability rates calculated by the task applicability rate calculation unit 15 may be displayed to a person to be undergoing rehabilitation through a user interface. A person to be undergoing rehabilitation can select and determine one task from a plurality of proposed task candidates. Note the value of N can be given in advance, or a threshold value may be defined in advance, and the value of N can be set be to be equal to or greater than the threshold value that is defined in advance.

The approach determining unit 3 determines tasks which the person to be undergoing rehabilitation should perform based on the determined tasks or the applicability rates of a plurality of goal candidates. In the case where the determined tasks are input in the approach applicability rate calculation unit 16, the approach applicability rate calculation unit 16 included in the approach determining unit 3 calculates the applicability rates of the plurality of the approach candidates for the person to be undergoing rehabilitation based on the tasks determined by the task determining unit 5. By referring to an approach applicability rate calculation table like Table 10 configured in advance, the applicability rates of the approach candidates can be calculated as the applicability rates (r₁, r₂, . . . ) allocated to the approach corresponding to the tasks. FIG. 4 is a diagram showing an example of candidates for approach for the determined task candidates.

TABLE 10
Approach applicability calculation table

| Rule | | | | Applicability |
|---|---|---|---|---|
| ID | Cause body part | Task | Approach | rate |
| 1 | Leftside hip + | Task 1 | Approach 1 | r₁ |
| | right knee | Task 2 | Approach 2 | r₂ |
| | | Task 3 | Approach 3 | r₃ |
| 2 | Left knee + | Task 4 | Approach 4 | r₄ |
| | right knee | | | |
| . . . | . . . | . . . | . . . | . . . |

Further, the applicability rates of the approach candidates can be calculated by directly inputting the applicability rates of the plurality of task candidates calculated by the task applicability rate calculation unit 15 into the approach applicability rate calculation unit 16. In this case, since the applicability rates of a plurality of task candidates are input, the applicability rates of the task candidates can be the sum or the mean value of the applicability rates of the respective task candidates calculated.

As described above, the rehabilitation approach proposal apparatus according to the present example embodiment can appropriately assess the level of nursing care a person to be undergoing rehabilitation needs by referring to the current bodily state information in which the current bodily state of the person to be undergoing rehabilitation is expressed in terms of states and tilt directions of the external body parts of the person to be undergoing rehabilitation. Further, by proposing approaches to be taken by a person to be undergoing rehabilitation based on the current bodily state information of the person to be undergoing rehabilitation and the information about the outcomes desired to be achieved by the person to be undergoing rehabilitation, it is possible to implement measures to prevent impairment in the elderly people's physical health.

As described above, the extraction rules (Table 2), the goal applicability rate calculation table (Table 6), the task applicability rate calculation table (Table 8), and the approach applicability rate calculation table (Table 10) can be generated through the data learning algorithm. The training data is a combination of the desired outcomes, the cause body parts, the reason body parts, the goals, the tasks, and the approaches as shown in Table 11. The extraction rules can be generated by compiling the cause body parts based on the training data. The specific values of the applicability rates in the tables can be obtained by statistical analysis techniques such as maximum likelihood estimation, MAP estimation, EAP estimation, and the like.

TABLE 11
Training data

| Desired | Cause body | Reason body | | | |
|---|---|---|---|---|---|
| outcome | part | part | Goal | Task | Approach |
| Desired | Front part of | Front part of | Goal 1 | Task 1 | Approach 1 |
| outcome 1 | leftside hip + | leftside hip | | | |
| | back of left | | | | |
| | knee | | | | |
| Desired | Front part of | back of left | Goal 2 | Task 2 | Approach 2 |
| outcome 1 | leftside hip + | knee | | | |
| | back of left | | | | |
| | knee | | | | |
| Desired | Front part of | front of left | Goal 3 | Task 3 | Approach 3 |
| outcome 2 | leftside hip + | knee | | | |
| | front of left | | | | |
| | knee | | | | |
| Desired | Back of left | back of right | Goal 4 | Task 4 | Approach 4 |
| outcome 3 | knee + back | knee | | | |
| | of right knee | | | | |
| . . . | . . . | . . . | . . . | . . . | . . . |

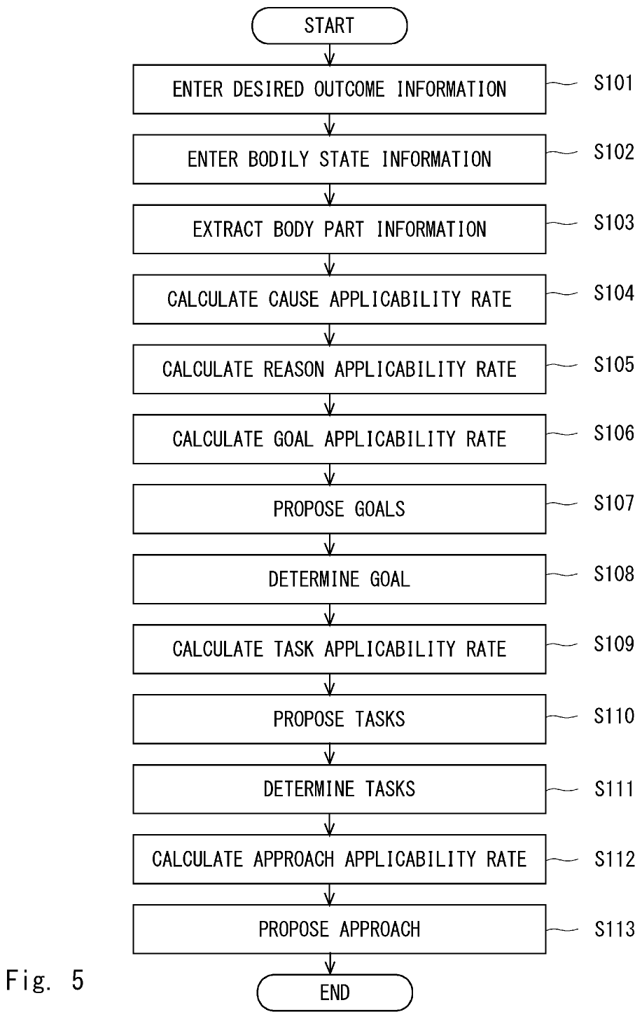

Next, by referring to the flowchart shown in FIG. 5, a rehabilitation approach proposal method according to an Example will be described. First, a desired outcome is selected by the desired outcome information input unit 2 (Step S101). As described above, the desired outcome candidates are listed as shown in FIG. 3. A person to be undergoing rehabilitation can select one desired outcome candidate from a plurality of desired outcome candidates through a user interface.

Then, the current bodily state information of the person to be undergoing rehabilitation is selected by the bodily state input unit 1 (Step S102). This current bodily state is expressed by all or at least one of the 18 external body parts of a person to be undergoing rehabilitation and the tilt of the external body parts in seven directions. A person to be undergoing rehabilitation can input his/her current bodily state through a user interface. Note that the order of input of the desired outcomes and the bodily state are not limited to this order.

Next, the body part information extraction unit 11 performs extraction of body part information of the selected cause body parts and the selected reason body parts (Step S103). The cause body parts are combination of external body parts that are determined in advance according to the rules, and the reason body parts include at least one external body part included in the cause body parts. Extraction of external body parts are implemented as many time as the number of the rules.

Then, the cause applicability rate calculation unit 13 calculates the applicability rate of each possible tilt state of respective extracted cause body parts (Step S104). For one rule, combinations of the extracted external body parts and the tilt states thereof are input in the cause applicability rate calculation unit 13 and a list of applicability rates of the combination of the external body parts and the possible tilt states thereof are output. This calculation of applicability rates is performed for each rule. That is, a list including applicability rates of the cause body parts is output as many as the number of rules.

Further, the reason applicability rate calculation unit 12 calculates the applicability rate of each possible tilt state of the extracted reason body parts (Step S105). In Step S105, as in Step S104, a list in which the applicability rates of the tilt states of the reason body parts is listed is output for the respective rules. Note that the order of calculation of the applicability rates performed in Step S104 and Step S105 are not limited to those described above, and the calculation in these steps may be performed at the same time.

Next, the goal applicability rate calculation unit 14 calculates the applicability rates of the plurality of goal candidates based on the selected desired outcome and the list in which the applicability rates of the tilt states of the cause body parts are listed (Step S106). In Step S106, a list in which the applicability rates of the goal candidates are listed is output in accordance with the respective rules. That is, here also, a list in which the applicability rates of the goal candidates are listed as many as the number of rules is output.

Then, the goal determining unit 4 performs proposal of the goals to be achieved based on the list in which applicability rates of each of the goal candidates are listed (Step S107). Across all the rules, the N-number of the goal candidates having the top N-number of applicability rates are displayed to a person to be undergoing rehabilitation through a user interface. For example, the goal candidates having the top five applicability rates are displayed in the descending order of the applicability rates. Then, the person to be undergoing rehabilitation selects and determines the goals to be achieved from the displayed goal candidates through a user interface (Step S108). Note that the goal determining unit 4 may automatically determine, without proposing the goal candidates, the goals based on the applicability rates of the goal candidates. Further, in the case where there is no goal which a person to be undergoing rehabilitation wants to select in the displayed goal candidates, a goal candidates that is not displayed can be selected.

Next, the task applicability rate calculation unit 15 calculates the applicability rates of the plurality of task candidates based on the determined goals and the list in which the applicability rates of the tilt states of the reason body parts are listed (Step S109). In Step S109, similarity of Step S106, a list in which the applicability rates of the goal candidates are listed is output in accordance with the respective rules. That is, here also, a list in which the applicability rates of the goal candidates are listed as many as the number of rules is output.

Then, the task determining unit 5 performs proposal of the tasks to be performed based on the list in which applicability rates of each of the task candidates are listed (Step S110). Across all the rules, the N-number of the goal candidates having the top N-number of applicability rates are displayed to a person to be undergoing rehabilitation through a user interface. For example, the goal candidates having the top five applicability rates are displayed in the descending order of the applicability rates. Then, the person to be undergoing rehabilitation selects and determines the tasks to be performed from the displayed task candidates through a user interface (Step S111). Note that task determining unit 5 may automatically determine, without proposing any task candidates, the tasks to be performed based on the applicability rates of the task candidates. Further, in the case here there is no task which the person to be undergoing rehabilitation desires to select among the displayed task candidates, the person to be undergoing rehabilitation can select task candidates that are not displayed.

Then, the approach applicability rate calculation unit 16 calculates the applicability rates of the plurality of the approach candidates for the person to be undergoing rehabilitation based on the determined tasks (Step S112). Note that in the present Example, since the approach is uniquely determined for the tasks, the approaches to be taken in view of the applicability rates of the selected tasks may be listed and the generated list may be output. The approach determining unit 3 displays the list of the approach candidates through a user interface (Step 113). Note that in the case where the approaches are duplicated, the duplicate items are deleted before displaying the list.

As described above, a rehabilitation approach proposal apparatus according to the present example embodiment can appropriately assess the level of nursing care a person to be undergoing rehabilitation needs by referring to the current bodily state information in which the current bodily state of the person to be undergoing rehabilitation is expressed in terms of the states and tilt directions of the external body parts of the person to be undergoing rehabilitation. By referring to the bodily state information appropriately assessed as described above, a rehabilitation approach proposal apparatus according to the present example embodiment can determine the goals matching the current bodily state of the person to be undergoing rehabilitation. Further, by determining the goals, the tasks, and the approaches step-by-step using the bodily state information appropriately assessed as described above, the rehabilitation approach proposal apparatus according to the present example embodiment can propose a suitable approach for in view of the bodily state of the person to be undergoing rehabilitation, the desired outcomes for the person to be undergoing rehabilitation, the tasks to be performed by the person to be undergoing rehabilitation, and the approaches to be taken by the person to be undergoing rehabilitation.

As described above, based on the goals, the tasks, and the approaches determined as described above by the rehabilitation approach proposal apparatus, any staff member who is not engaged in rehabilitation profession can take a suitable approach to a person to be undergoing rehabilitation. Further, a rehab professional staff member can perform rehab therapy more appropriately and easily based on the goals, the tasks, and the approaches proposed by the rehabilitation approach proposal apparatus.

Further, according to the present Example, by selecting the desired outcomes and the bodily state from a limited number of choices, even family members of a person to be undergoing rehabilitation or a staff member who is not engaged in rehabilitation profession and have no professional knowledge and experience in performing rehabilitation can determine the goals, the tasks, and the approaches depending on the bodily state of the person to be undergoing rehabilitation without having to assess the nursing care state in detail, whereby it is possible to contribute to prevention of impairment in the physical heath of the person to be undergoing rehabilitation.

Note that the functional blocks shown in the figures that perform various processing can be configured of a CPU, a memory, and other circuits in terms of hardware configuration. Further, the present disclosure can implement the aforementioned processing by causing a processor to execute a computer program. Therefore, these functional blocks can be implemented by a hardware configuration, a software configuration, or a combination thereof, and it is not to be limited to any one of them.

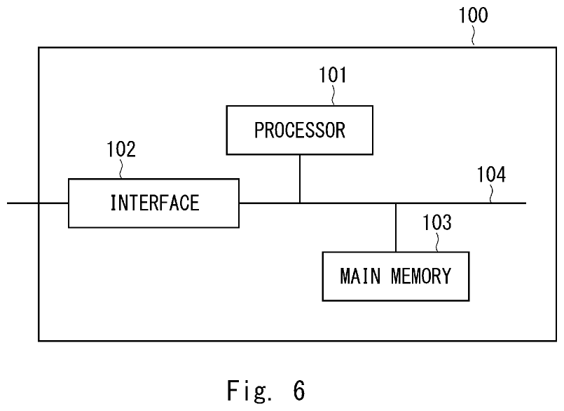

FIG. 6 is a block diagram showing an example of a hardware configuration of a rehabilitation approach proposal apparatus according to an Example. Here, the rehabilitation approach proposal apparatus 10 includes an information processing apparatus (a computer) 100. As shown in FIG. 6, the computer 100 includes a processor 101, an interface 102, and a main memory 103, and they are connected with one another through a bus 104. However, the configuration shown in FIG. 6 is not intended to limit the configuration of the rehabilitation approach proposal apparatus 10.

The interface 102 corresponds to the aforementioned user interface such as a display apparatus or an input apparatus that is used by a user, who is a person to be undergoing rehabilitation or a caregiver, in performing data exchange with the computer 100. A display apparatus displays the candidates list shown in the aforementioned Table 3 and a list of the goals, the tasks, and the approaches that are based on the calculated applicability rates. Examples of the input apparatus includes a keyboard, a mouse, and the like. Further, a touch panel in which a display apparatus and an input apparatus are integrally configured may be used. The processor 101 input and output various types of information through the interface 102. The main memory 103 includes a RAM (Random Access Memory), a ROM (Read Only Memory), and an auxiliary storage apparatus (e.g. a hard disk). The main memory 103 store programs and data to be executed the processor 101. Programs stored in the main memory 103 include an instruction set for causing the processor 101 to execute the various processings.

The processor 101 execute the programs store in the main memory 103. The processor 101 is, for example, a CPU (Central Processing Unit). As described above, the respective functions can be realized by causing the processor 101 execute the program for performing the aforementioned processings.

In the aforementioned examples, a program can be stored and provided to a computer using any type of non-transitory computer readable media. Non-transitory computer readable media include any type of tangible storage media. Examples of non-transitory computer readable media include magnetic storage media (e.g. floppy disks, magnetic tapes, hard disk drives), optical magnetic storage media (e.g. magneto-optical disks), CD-ROM (Read Only Memory), CD-R, CD-R/W, semiconductor memories (e.g. mask ROM, PROM (Programmable ROM), EPROM (Erasable PROM), flash ROM, and RAM (Random Access Memory), etc.). The program may be provided to a computer using any type of transitory computer readable media. Examples of transitory computer readable media include electric signals, optical signals, and electromagnetic waves. Transitory computer readable media can provide the program to a computer via a wired communication line such as electric wires and optical fibers or a wireless communication line.

As described above, according to the example embodiment, even family members of a person to be undergoing rehabilitation or staff members who are not engaged in rehabilitation profession can easily assess the nursing care state of a person to be undergoing rehabilitation and set the goals and the tasks depending on the bodily state of the person to be undergoing rehabilitation and propose a suitable approach that can be taken at an own pace of the person to be undergoing rehabilitation at home, whereby it is possible to contribute to prevention of impairment in the physical heath of the person to be undergoing rehabilitation.

Note that the present disclosure is not limited to the above-described example embodiments, and can be appropriately changed without departing from the gist of the present disclosure. For example, in the aforementioned Examples the approach to be taken depends on only the tasks and therefore the approach to be taken is uniquely determined and the approach to be taken is also uniquely determined. The present disclosure is not to be limited to the aforementioned example embodiments and determination of the approaches to be taken can be extended to depend on the desired outcomes and the bodily state of a person to be undergoing rehabilitation, or the cause body parts or the reason body parts generated from the bodily state.

The present disclosure has been described above with reference to the drawings but the present disclosure is not to be limited to the above-described example embodiments. The configuration and details of the present disclosure can be changed in various ways without departing from the scope of the present disclosure.

This application claims priority based on Japanese Patent Application No. 2020-200133 filed on Dec. 2, 2020, the disclosure of which is herein incorporated by reference in its entirety.

### REFERENCE SIGNS LIST

1 Bodily State Information Input Unit
2 Desired Outcome Information Input Unit
3 Approach Determining Unit
4 Goal Determining Unit
5 Task Determining Unit
10 Rehabilitation Approach Proposal Apparatus
11 External Body Part Information Extraction Unit
12 Reason Applicability Rate Calculation Unit
13 Cause Applicability Rate Calculation Unit
14 Goal Applicability Rate Calculation Unit
15 Task Applicability Rate Calculation Unit
16 Approach Applicability Rate Calculation Unit
100 Computer
101 Processor
102 Interface
103 Main Memory
104 Bus

What is claimed is:

1. A rehabilitation approach proposal apparatus comprising:
at least one memory storing instructions; and
at least one processor configured to execute the instructions to:
input bodily state information of a person to be undergoing rehabilitation as bodily state information, the bodily state information being expressed by current states and tilts of external body parts of the person to be undergoing rehabilitation;
select and inputting a desired outcome of rehabilitation from a plurality of desired outcome candidates as desired outcome information of the person to be undergoing rehabilitation; and
determine a rehabilitation approach to be taken by the person to be undergoing rehabilitation based on the bodily state information and the desired outcome information.

2. The rehabilitation approach proposal apparatus according to claim 1, wherein at least one processor is further configured to execute the instructions to:
extract information on a cause body part and a reason body part from the bodily state information, the cause body part being a predetermined combination of external body parts that are causing the person to be undergoing rehabilitation to have a current body state and the reason body part including at least one of the external body parts included in the cause body part and in which a reason for which the person to be undergoing rehabilitation has the current bodily state is found; and
determine a goal that the person to be undergoing rehabilitation desires to achieve after undergoing rehabilitation based on the information on the cause body part and the desired outcome information.

3. The rehabilitation approach proposal apparatus according to claim 2, wherein at least one processor is further configured to execute the instructions to:
calculate an applicability rate of each possible tilt of the cause body part, and
determine the goal to be achieved based on an applicability rate of the goal candidate from a plurality of goal candidates that the person to be undergoing rehabilitation desires to achieve after undergoing rehabilitation using the desired outcome information and information on the calculated applicability rate of each possible tilt of the cause body part.

4. The rehabilitation approach proposal apparatus according to claim 3, wherein at least one processor is further configured to execute the instructions to:
calculate the applicability rate of the possible tilt of each cause body part by multiplying the applicability rates of the possible tilts of the respective external body parts included in the cause body part.

5. The rehabilitation approach proposal apparatus according to claim 3, wherein at least one processor is further configured to execute the instructions to:
determine a task to be performed when undergoing rehabilitation based on the determined goal to be achieved or the applicability rate of each goal candidate of the plurality of goal candidates, and the cause body part.

6. The rehabilitation approach proposal apparatus according to claim 5, wherein at least one processor is further configured to execute the instructions to: calculate an applicability rate of each possible tilt of the reason body part,
calculate the applicability rates of each of the plurality of task candidates to be performed by the person to be undergoing rehabilitation using information on the determined task to be performed or information on applicability rate of each of the plurality of goals and the calculated applicability rate of the each possible tilt of the reason body part and determine the task to be performed by the person to be undergoing rehabilitation from the plurality of the task candidates, and
determine a task which the person to be getting undergoing rehabilitation should perform based on the determined task or the applicability rate of each goal of the plurality of goal candidates.

7. The rehabilitation approach proposal apparatus according to claim 1, wherein the bodily state information is expressed by at least one external body part of 18 external body parts of the person to be undergoing rehabilitation and tilt of the respective external body parts in seven directions.

8. The rehabilitation approach proposal apparatus according to claim 7, wherein
the 18 external body parts are leftside and rightside of the cervical, left and right shoulders, leftside and rightside of the hip, left and right hip joints, left and right knees, left and right ankles, left and right elbows, left and right wrists, and left and right hand fingers, and
the seven directions are left, right, forward, backward, up, down, and no tilt.

9. A rehabilitation approach proposal method comprising determining a rehabilitation approach to be taken by a person to be undergoing rehabilitation based on bodily state information of the person to be undergoing rehabilitation and desired outcome information, the body state information being expressed by current states and tilts of external body parts of the person to be undergoing rehabilitation and the desired outcome information being selected from a plurality of desired outcome candidates.

10. A non-transitory computer readable medium storing a rehabilitation approach proposal program for causing a computer to execute processes for determining a rehabilitation approach to be taken by a person to be undergoing rehabilitation based on bodily state information and desired outcome information, the body state information being expressed by current states and tilts of external body parts of the person to be undergoing rehabilitation and the desired outcome information being selected from a plurality of desired outcome candidates.
